(19) United States
(12) Patent Application Publication
(10) Pub. No.: US 2012/0116136 A1
(43) Pub. Date: May 10, 2012

(54) DEVICE FOR DISTRIBUTING A POLYPHASE MIXTURE COMPRISING A JET BREAKER TRAY WITH A SEPARATING ELEMENT

(75) Inventors: Frederic AUGIER, Saint Symphorien D Ozon (FR); Frederic BAZER-BACHI, Saint Genis-Laval (FR); Christophe BOYER, Charly (FR); Emilie GAGNIERE, Villeurbanne (FR)

(73) Assignee: IFP ENERGIES NOUVELLES, RUEIL-MALMAISON CEDEX (FR)

(21) Appl. No.: 13/224,594

(22) Filed: Sep. 2, 2011

(30) Foreign Application Priority Data
Sep. 3, 2010 (FR) 10/03.531

(51) Int. Cl. C07C 5/10; B01J 8/02

(52) U.S. Cl. 585/266; 422/215

(57) ABSTRACT

The present invention concerns a device for distributing a polyphase mixture constituted by at least one gas phase and at least one liquid phase, said mixture being in downflow mode passing through at least one bed of solid particles, and said device comprising at least one tray (1) located above a bed of solid particles, a plurality of mixing channels (2) for said liquid and gas phases, a dispersive system (3) of the jet breaker tray type (3) with a controlled porosity provided with flanges (36) over at least a portion of its perimeter, disposed beneath the mixing channels (2) and above the bed of solid particles, said distribution device being characterized in that the dispersive system (3) comprises at least one separating element (32).

The present invention relates to the field of the distribution of polyphase fluids in catalytic reactors and more particularly to a device that can be used to optimize the distribution of fluids in catalytic reactors of the fixed bed type, functioning in downflow mode, in applications of the gas oil hydrotreatment type and all hydrogenation operations functioning in gas-liquid trickle flow mode.

In particular, the present invention is applicable to the field of gas/liquid distributors such as, for example, those employed to carry out hydrocracking, hydrotreatment, hydrodesulphurization, hydrodemetallization, hydrodenitrogenation, selective or total hydrogenation, the selective hydrogenation of steam cracked gasoline, the hydrogenation of aromatic compounds in aliphatic and/or naphthenic cuts, and the hydrogenation of olefins in aromatic cuts.

It is also of application in carrying out other reactions necessitating good mixing of a gas phase and a liquid phase, for example partial or total oxidation reactions, amination reactions, acetyloxidation reactions, ammoxidation reactions and halogenation reactions, in particular chlorination.

In general, then, the distribution device is disposed in a vessel or reactor comprising an inlet for a liquid fluid and an inlet for a gaseous fluid, and containing at least one bed, for example of granular solids.

That device may be disposed:
either at the head of the vessel above the bed;
or at the outlet from one bed with a supply over the whole section of the vessel, covering the subsequent bed.

In order to improve the distribution of fluids using said devices, one possibility employed in the prior art consists, for example, of using distributor trays comprising a plurality of mixing channels dedicated to the passage of gas and liquid. Those mixing channels may be of various types and are positioned in various configurations over the tray. Such devices have been described in patent applications FR 2 807 676, FR 2 745 202, FR 2 853 260 or US 2007/0241467.

The disadvantage of that type of mixing channel lies in the fact that the flow leaving the mixing channel forms a relatively concentrated two-phase jet, which is problematic since the liquid is not sprinkled over the whole section of the column. In order to overcome that problem, the spacing between the mixing channels is relatively small (generally between 80 and 200 mm), which considerably increases the number of mixing channels and thus increases the overall cost of the distributor tray.

Another solution to improving sprinkling of the bed is to position jet dispersion elements below the mixing channels. Several types of dispersion elements may be used. Insert type elements are often attached to each mixing channel in order to disperse the jet homogenously over a wider angle below the mixing channel, as described in patent applications EP 2 075 056 and US 2010/0019061.

That type of solution is effective, but it requires a certain distance to be maintained between the mixing channel and the top of the bed of particles so that the jet can sprinkle the bed over a wide area. Ideally, this distance must allow the jets formed by neighbouring mixing channels to join up. Further, that solution is fairly costly due to the large number of dispersion elements to be manufactured.

An alternative solution described in patent FR 2 807 673 consists of positioning jet breaker type dispersion elements beneath the tray which are common to several mixing channels (or even common to an entire row). Conventionally, screens or perforated trays are employed. It is important that the various elements used remain discrete so that the gas can circulate freely between the dispersion elements without being constrained to pass through the elements per se. The gas-liquid jet impinges on the screen and is dispersed over its surface before raining down onto the bed of particles.

Patent FR 2 832 075 describes an improvement to that device which consists of adding flanges solely to the perimeter of the screens. Said flanges allow liquid to remain at the screen and not to overflow preferentially at the screen periphery. That type of device has a number of advantages:
it is less expensive than inserts and also means that the spacing between the mixing channels can be increased;
the flow is distributed over the column section and thus there is no need for a jet formation distance to be allowed for beneath the screens.

The disadvantage of that type of device is that its function may be altered if the tray is not perfectly straight. If the tray is slightly inclined in one direction (flexing of tray under its own weight, mounting tolerances, being out of horizontal, etc), the screen may then direct a portion of the liquid phase in one direction and act as a drain. This therefore results in poor distribution of liquid in the bed and a reduction in reaction performance (loss of conversion and/or selectivity). That problem is encountered with riser type mixing channels but also with vapour lift type mixing channels or bubble cap type channels.

Thus, the present invention aims to overcome one or more of the disadvantages of the prior art by proposing a distribution device comprising a jet breaker tray, which can limit or prevent poor distribution of the liquid flow in the case of the jet breaker tray being out of the horizontal.

To this end, the present invention proposes a device for distributing a polyphase mixture constituted by at least one gas phase and at least one liquid phase, said mixture being in downflow mode passing through at least one bed of solid particles, and said device comprising at least one tray located above a bed of solid particles, a plurality of mixing channels for said liquid and gas phases, a dispersive system of the jet breaker tray type with a controlled porosity provided with flanges over at least a portion of its perimeter, disposed beneath the mixing channels and above the bed of solid particles, said distribution device being characterized in that the dispersive system comprises at least one separating element.

In one embodiment of the invention, the separating element is formed by a plate positioned perpendicular to the jet breaker tray.

In one embodiment of the invention, the height of the separating element is in the range 50% to 100% of that of the flange of the jet breaker tray.

In one embodiment of the invention, the separating element is in the shape of a planar rectangular parallelepiped.

In one embodiment of the invention, the separating element is solid or pierced or porous.

In one embodiment of the invention, the separating element closes in the range 40% to 100% of the cross section of the jet breaker tray.

In one embodiment of the invention, the separating element is positioned directly beneath a mixing channel.

In one embodiment of the invention, the separating element is positioned beneath a mixing channel offset with respect to said mixing channel so as to be positioned between two mixing channels.

In one embodiment of the invention, the dispersive system comprises several separating elements.

In one embodiment of the invention, the separating elements are disposed in order to separate between 1 and 10 mixing channels.

In one embodiment of the invention, the distribution of the separating elements varies as a function of their position on the jet breaker tray.

In one embodiment of the invention, the separating element is attached by an attachment system comprising means for obstructing a portion of the jet breaker tray.

In one embodiment of the invention, the obstruction means are formed by a plate.

In one embodiment of the invention, the plate may be square, rectangular, round or oval in shape.

In one embodiment of the invention, the mixing channels are risers.

In one embodiment of the invention, the mixing channels are vapour lifts.

In one embodiment of the invention, the mixing channels are bubble caps.

The invention also concerns the use of a device as described above in a reactor suitable for hydrotreatment or hydrogenation or oxidation.

Other characteristics and advantages of the invention will be better understood and will become apparent from the following description, made with reference to the accompanying drawings and given by way of example, in which:

### BRIEF DESCRIPTION OF THE DRAWINGS

FIG. 1 is a diagrammatic perspective view of the distribution device of the invention positioned under mixing channels;

FIG. 2 is a diagrammatic top view of the distribution device of the invention;

FIG. 3 is a diagrammatic top view of a variation of the distribution device of the invention;

FIG. 4 is a diagrammatic top view of a variation of the distribution device of the invention;

FIG. 5 is a diagrammatic top view (5a) and side view (5b) of a variation of the distribution device of the invention;

FIG. 6 is a diagrammatic side view of an example of the use of a distribution device of the invention.

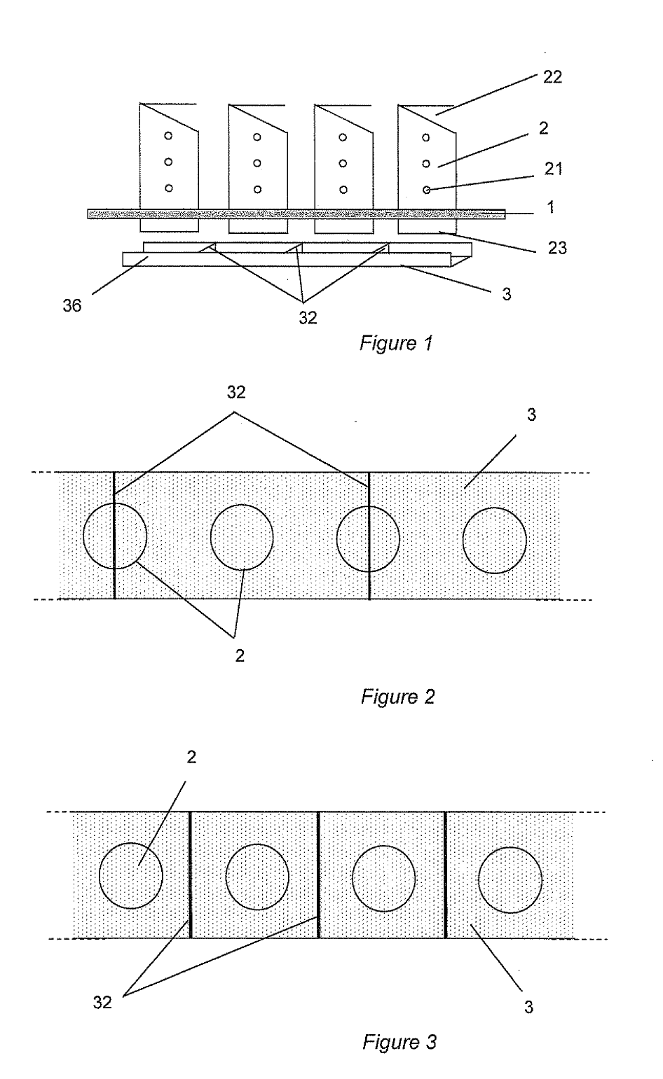

As can be seen in FIGS. 1 to 5, the device of the present invention may comprise a plurality of mixing channels such as risers 2 having at least one upper section of flow 22, for example a taper, at its upper portion, and a lower section of flow 23. These mixing channels (illustrated as circles in FIGS. 2, 3, 4 and 5a) may comprise a plurality of holes 21 allowing the passage of liquid. Beneath the tray 1, a jet breaker tray type dispersive system 3 receives the polyphase mixture formed in the mixing channels 2.

The height of the mixing channels is usually in the range 100 to 500 millimetres (mm), preferably in the range 200 to 400 mm.

In one embodiment of the invention, the mixing channels may also be vapour lifts. The vapour lift type device (described in patents U.S. Pat. No. 7,600,742 and U.S. Pat. No. 5,942,162) is constituted by tubes forming an M-shaped circuit for circulation of fluid. The term “lift” is used because the vapour initially rises in the device via the outer tubes and then descends via the central tube.

In another embodiment of the invention, the mixing channels may also be bubble caps. The mixing channel tubes project beyond the tray 1 by a height which normally is between 10 and 200 mm and is often between 25 and 50 mm. Usually, the mixing channel extends below the distributor tray over a length which is less than or equal to the distance between the outlet 23 from a mixing channel and the jet breaker type tray 3.

The portion of the mixing channels 2 disposed above the tray is pierced with holes 21 or slots over its periphery at one or more levels, preferably at least two levels.

The means for ensuring dispersion of the two-phase or polyphase mixture formed in the mixing channel is a jet breaker tray type dispersive system 3 located beneath and in the proximity of the section of flow 23 of the mixing channels. This jet breaker dispersive system is in the form of a jet breaker tray and may either have holes or be porous. When the tray has holes, the holes may have different sizes depending on their position on the tray. As an example, the further the holes are from the mixing channel, the larger their diameter could be, or vice versa.

The distance between the outlet 23 from the mixing channel and the jet breaker tray 3 usually varies from 5 to 500 mm, usually 10 to 200 mm and preferably 50 to 100 mm. The jet breaker tray is usually constituted by several separated elements disposed at different heights (with respect to the outlet 23 from the mixing channels) but for which the totality of the surfaces covers the section of the reactor. This difference in distance between the various elements of the jet breaker tray and the outlet 23 from the mixing channels means that a free section of flow can be left for the passage of gas. The jet breaker tray 3 is located at a distance from the bed of granular solids in order to conserve the mixture formed inside said mixing channels and leaving said mixing channels via said lower sections of flow until it is distributed into the bed of granular solids. This distance is normally in the range 0 to 500 mm, preferably in the range 1 to 100 mm.

The jet breaker tray may be suspended at the tray 1 or at the lower end of the mixing channels 2.

The jet breaker tray 3 also comprises a flange 36 that can maintain a level of liquid over the whole surface of the high throughput screens. This flange 36 is disposed over the whole of the jet breaker tray 3, these flanges possibly themselves being porous. The height of the flanges 36 may be in the range 0.1 to 1 times the diameter of the channels, for example in the range 2 to 50 mm. They may themselves have a porosity in the range 0 to 80%. They may or may not be inclined to the vertical, and their inclination is generally in the range −40° to +60°, preferably in the range −30° to +45°, the values of these angles being with respect to the vertical, with positive values corresponding to flanges inclined outwardly of the dispersive system and negative values corresponding to flanges inclined inwardly of the dispersive system. Clearly, when dispersive systems belonging to different horizontal planes are provided with flanges, the distance separating these horizontal planes must be greater than the height of the flanges. The flanges 36 may concern just a portion of the dispersive systems, the other portion not having such flanges. It is often preferable to provide the dispersive systems located on the planes closest to the granular solid with flanges. In certain cases, it may even be advantageous for a given dispersive system to have flanges over only a portion of its perimeter. The precise geometric shape of these flanges may vary; in particular, the upper end of the flanges may be curved inwardly. In the vicinity of the flange of a dispersive system, the porosity of the dispersive system may be zero or identical to the remainder of the surface of the dispersive system 3. The term “in the vicinity of the flange of a dispersive system” means the zone located at a distance of 30 mm or less from the flange and preferably 20 mm or less from the flange.

One of the functions of said flanges and their near-zero porosity is to retain certain impurities that may be contained in the liquid feed, particularly when it is constituted by heavy hydrocarbons such as cuts with a boiling point of more than 350° C., as is the case with units for the hydrotreatment of heavy gas oil type cuts.

In this case, the zone in the vicinity of the flanges gradually becomes laden with impurities, thereby preventing contamination of the bed of granular solids.

The subject matter of the present invention consists of positioning at least one separating element 32, also termed a baffle, across the jet breaker trays 3 to limit or prevent the liquid flow leaving a mixing channel 2 from being driven to a distance far from that channel by a preferential gravity flow over the jet breaker tray. This means that in the case in which the jet breaker tray 3 is not completely horizontal for the reasons mentioned above, the separating element will prevent the liquid from accumulating under gravity at a single location of the jet breaker and then from overflowing in an irregular manner onto the particle bed. The jet breaker tray may comprise one or more separating elements 32. These separating elements may be formed by plates positioned perpendicular to the jet breaker tray, i.e. perpendicular to the bottom of the tray. Their height is generally in the range 20% to 100% that of the flange 36 of the jet breaker tray 3, preferably in the range 50% to 90%. They are generally disposed in the plane transverse to the jet breaker tray 3.

In accordance with one embodiment of the invention, the separating elements 32 are orientated in a plane perpendicular to the longest flange of the tray 3.

These separating elements 32 may be disposed in different manners. They may be positioned directly beneath a mixing channel 2 so as to disperse its flow (FIG. 2). They may be positioned beneath the mixing channels but offset with respect to the mixing channels so that they are positioned between two mixing channels (FIG. 3).

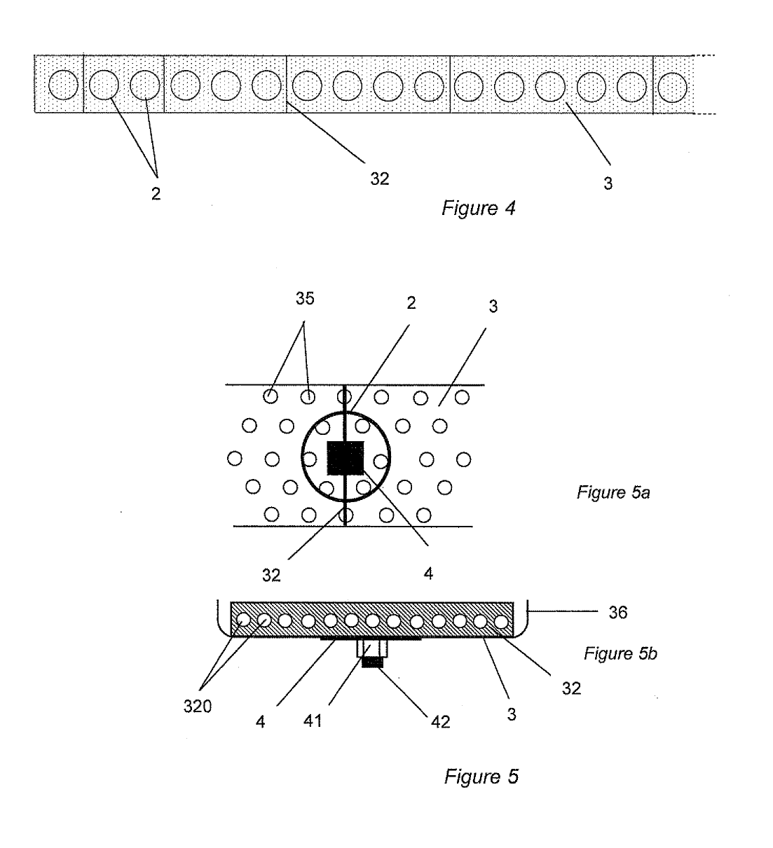

In certain cases, mechanical flexing of the jet breaker trays 3 is not homogenous over the section of the column or reactor. This flexing greatly distorts the tray close to the flange and deforms it less at the centre. It is thus possible to distribute the dispersion elements as a function of their position with respect to the flange. The distribution of the separating elements 32 thus varies as a function of their position on the jet breaker tray 3. As an example, the separating elements 32 may be disposed in an arithmetical manner as illustrated in FIG. 4. In this case, the separating elements 32 are not disposed between the mixing channels 2 in a regular manner but so as to separate a different number of mixing channels 2, for example every 1, 2, 3, 4 etc mixing channels 2. They may also be disposed in greater numbers at the ends of the jet breaker tray 3, for example for every mixing channel 2 in these zones, and every 2, 3, 4 etc mixing channels 2 in the middle.

These examples are not in any way limiting and any position for the separating elements 32 may be envisaged. As an example, the separating elements 32 may be disposed every 1 to 10 mixing channels, preferably every 1 to 5 mixing channels.

These separating elements 32 may be in the shape of a planar rectangle or any other shapes adapted to the jet breaker tray 3 used. The separating elements 32 may be solid (FIG. 1), pierced (FIG. 5b) or porous in order to partially or completely seal off the cross section of the jet breaker tray 3. Irrespective of their shape, the separating elements close off in the range 40% to 100% of the cross section of the tray, preferably in the range 50% to 100%. The cross section corresponds to the section orientated along the longitudinal plane of the separating element 32.

The separating elements 32 are attached to the jet breaker tray and to the flange by conventional attachment means.

The system for attaching the separating elements 32 to the jet breaker tray 3 may also be designed to obstruct a portion of the jet breaker tray 3 (FIG. 5a). The attachment system may, for example, comprise fixing means comprising means for obstructing said type of plate 4 which will plug one or more holes of the jet breaker tray 3. The plate may be round, oval, square or rectangular in shape or it may have a different shape. The shape and size of the plate is selected as a function of the number of holes or the surface area of the jet breaker tray 3 to be obstructed. Such a plate may, for example, obstruct one or more holes. The plate may be welded or fixed to the jet breaker tray 3 with an attachment system of the screw 42 and bolt 41 type (as illustrated in FIG. 5b), or any other attachment system suitable for the device of the invention.

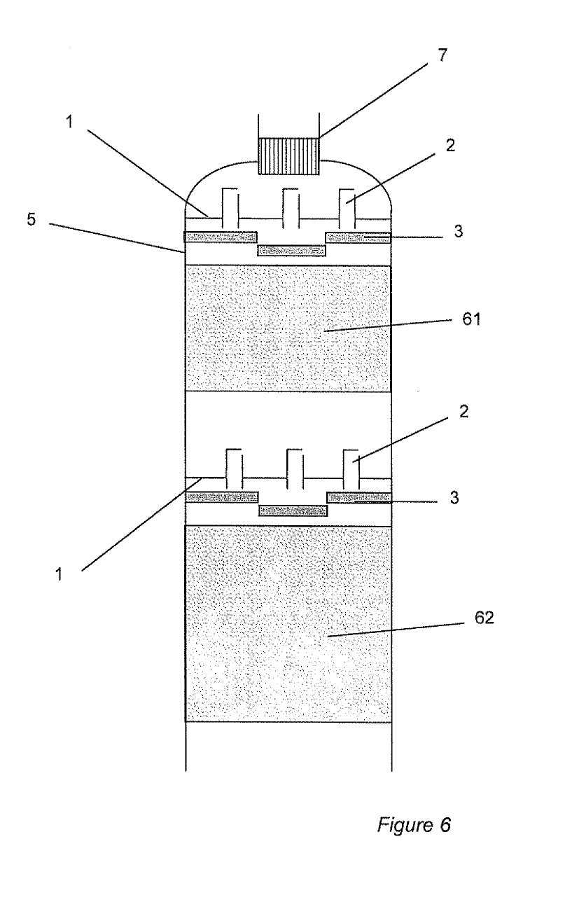

FIG. 6 illustrates a simplified implementation of the distribution device of the invention. The description given below by way of an example of an application concerns a distribution system used in a reactor operating in downflow mode adapted for hydrotreatment or hydrogenation. The reactor comprises a vessel 5 comprising a pre-distributor 7 in its upper portion or reactor head. The mixture distributed by the pre-distributor 7 flows in downflow mode to the distributor tray 1 which is located above a first bed of granular solids 61 or catalytic bed. The tray comprises a plurality of mixing channels 2 opening onto the dispersive jet breaker type tray 3. After its passage through the bed of granular solids 61, the two-phase mixture is re-distributed directly onto a second bed of granular solids 62 after having passed through a second device in accordance with the present invention.

The present invention is not limited to the details given above; other specific types of embodiments are permitted without going beyond the scope of application of the invention. As a consequence, the present embodiments should be considered to be by way of illustration and may be modified without, however, going beyond the scope as defined in the claims.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding French application Ser. No. 10/03531, filed Sep. 3, 2010, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A device for distributing a polyphase mixture constituted by at least one gas phase and at least one liquid phase, said mixture being in downflow mode passing through at least one bed of solid particles, and said device comprising at least one tray (1) located above a bed of solid particles, a plurality of mixing channels (2) for said liquid and gas phases, a dispersive system (3) of the jet breaker tray type (3) with a controlled porosity provided, over at least a portion of its perimeter, with flanges (36), disposed beneath the mixing channels (2) and above the bed of solid particles, said distribution device being characterized in that the dispersive system (3) comprises at least one separating element (32).

2. A device according to claim 1, characterized in that the separating element (32) is formed by a plate positioned perpendicular to the jet breaker tray (3).

3. A device according to claim 1, characterized in that the height of the separating element (32) is in the range 50% to 100% of that of the flange (36) of the jet breaker tray (3).

4. A device according to claim 1, characterized in that the separating element (32) is in the shape of a planar rectangular parallelepiped.

5. A device according to claim 1, characterized in that the separating element (32) is solid or pierced or porous.

6. A device according to claim 1, characterized in that the separating element (32) closes in the range 40% to 100% of the cross section of the jet breaker tray (3).

7. A device according to claim 1, characterized in that the separating element (32) is positioned directly beneath a mixing channel (2).

8. A device according to claim 1, characterized in that the separating element (32) is positioned beneath a mixing channel (2) which is offset with respect to said mixing channel (2) so as to be positioned between two mixing channels (2).

9. A device according to claim 1, characterized in that the dispersive system comprises several separating elements (32).

10. A device according to claim 8, characterized in that the separating elements (32) are disposed in order to separate between 1 and 10 mixing channels (2).

11. A device according to claim 1, characterized in that the distribution of the separating elements (32) varies as a function of their position on the jet breaker tray (3).

12. A device according to claim 1, characterized in that the separating element is attached by an attachment system comprising means (4) for obstructing a portion of the jet breaker tray (3).

13. A device according to claim 12, characterized in that the obstruction means are formed by a plate (4).

14. A device according to claim 12, characterized in that the plate (4) can be square, rectangular, round or oval in shape.

15. A device according to claim 1, characterized in that the mixing channels are risers.

16. A device according to claim 1, characterized in that the mixing channels (2) are vapour lifts.

17. A device according to claim 1, characterized in that the mixing channels (2) are bubble caps.

18. A process for hydrotreatment or hydrogenation or oxidation, comprising subjecting a hydrocarbon feed to said process in a device according to claim 1.
